(19) United States
(12) Patent Application Publication
(10) Pub. No.: US 2020/0093967 A1
(43) Pub. Date: Mar. 26, 2020

(54) IMPLANTABLE DEVICE FOR MODULATING LOCALIZED PH AT IMPLANTATION SITE

(71) Applicant: THE REGENTS OF THE UNIVERSITY OF COLORADO, A BODY CORPORATE, DENVER, CO (US)

(72) Inventors: Jennifer Wagner, Broomfield, CO (US); Tina Govindarajan, Lafayette, CO (US); Michal Schafer, Franktown, CO (US); Karl Treadwell, Missoula, MT (US); Omid Jazaeri, Denver, CO (US); Rajan Gupta, Denver, CO (US)

(73) Assignee: THE REGENTS OF THE UNIVERSITY OF COLORADO, A BODY CORPORATE, DENVER, CO (US)

(21) Appl. No.: 16/582,757

(22) Filed: Sep. 25, 2019

Related U.S. Application Data
(60) Provisional application No. 62/736,392, filed on Sep. 25, 2018.

(51) Int. Cl. A61L 31/14; A61N 1/20; A61L 31/02; A61F 2/01

(52) U.S. Cl. CPC A61L 31/14; A61N 1/205; A61F 2250/0067; A61F 2/01; A61L 31/022

(57) ABSTRACT

Implantable devices disclosed herein may be configured to modulate the localized pH at an implantation site of the implantable device. By controlling, modulating, or otherwise adjusting the localized pH, various benefits can be achieved, such as controlling cell proliferation. The implantable device may include a body and one or more metallic features. Generally, the implantable device forms a galvanic cell such that a first metallic feature is configured to be preferentially oxidized to alter the localized pH environment in the vicinity of the implantable device.

### CROSS-REFERENCE TO RELATED APPLICATIONS

This application claims priority to and benefit of U.S. provisional patent application Ser. No. 62/736,392, filed on Sep. 25, 2018, and entitled “METHOD AND APPARATUS FOR CONTROLLING THE ENDOTHELIALIZATION OF IMPLANTABLE DEVICES,” the entire contents of which are incorporated herein by reference in their entirety for all purposes.

### FIELD

The present disclosure relates to implantable devices, and more particularly to devices, systems, and methods for modulating localized pH at the implantation site of implantable devices.

### BACKGROUND

Surface modification has proven an effective method of enhancing the performance of medical devices at their biological interfaces. Materials used in the manufacture of early medical devices were selected primarily based upon their availability for industrial applications. Not surprisingly, the surface properties of first-generation devices were not optimized for use in the human body.

For example, conventional inferior vena cava (IVC) filters are vascular filters that are inserted into the inferior vena cava, a large vein that carries deoxygenated blood into the right atrium of the heart, to prevent potentially life-threatening pulmonary emboli (PE), particularly in patients who are at high risk of developing PE and cannot be sufficiently anticoagulated. Although IVC filters are effective to reduce the incidence of PE, they are not intended to act as permanent replacements for pharmaceutical management of venous thromboembolism (VTE) and may cause significant long-term complications. For example, in August 2010, the FDA issued a warning describing 921 reports of complications involving IVC filters. Of these 921 reports, 328 involved device migration, 146 involved detachment of device components, 70 involved perforation of the Inferior Vena Cava, and 56 involved filter fracture. Most notably, there was a 20% increased risk of deep vein thrombosis (DVT) and vena cava occlusion leading to potential vascular collapse and death.

To mitigate the disadvantages of long-term use of IVC filters, IVC filters have been developed that may be implanted and later retrieved and removed, e.g. when the patient's risk of PE can be lowered by other means. Most commonly, retrievable IVC filters are fitted with any one of several devices, known to those skilled in the art, that allow the filter to be easily snared and pulled back into a sheath and then removed from the body, often via the jugular vein in the neck. Despite their advantages, however, retrievable IVC filters still suffer from several drawbacks. Perhaps the most significant problem afflicting retrievable IVC filters is that they tend to quickly become incorporated into the wall of the IVC with endothelial cells migrating from the IVC wall.

The body's response to implanted medical devices comprises a series of events, beginning with acute inflammatory response, followed by chronic inflammatory response, granulation tissue development, and foreign body reaction to implanted biomaterials. The intensity and time course of each depends upon factors such as the extent of injury, and the size, shape, topography, and chemical and physical properties of the implanted device. This process makes retrieval of the filter more difficult, especially if the filter has been placed for an extended period of time. In the period during which retrieval of the filter is still recommended, the removal of a filter that has undergone even a small degree of incorporation presents an increased risk of injury to the patient arising from disruption of the IVC wall.

Endothelialization presents difficulties in the use of medical devices other than IVC filters as well. For example, endothelialization of a surface, or part of a surface, of a vascular stent, stent graft, or cardiac pacing lead wires may be desirable or undesirable, depending on the application. Methods that promote a healthy endothelial layer may not only provide the means to decrease adverse events associated with current devices such as coronary and peripheral stents, structural heart devices, peripheral vascular grafts, and cardiac patches, but also enable other devices that have thus far proven unsuccessful clinically, such as small diameter grafts. In other words, depending on the specific surgical implantation, cell growth around the implanted device (e.g., endothelialization) may be desirable or undesirable.

### SUMMARY

In various embodiments, the present disclosure provides an implantable device configured to affect a localized pH at an implantation site. The implantable device generally includes, according to various embodiments a body and a first feature. The body may comprise a base metallic material and the first feature may be coupled to the body and may comprise a first metallic material. The first metallic material, according to various embodiments, has a lower electrode potential than the base metallic material such that the first metallic material is configured to be preferentially oxidized over the base metallic material.

In various embodiments, the first feature is anodic and the body is cathodic. In various embodiments, the first metallic material comprises at least one of magnesium, zinc, and aluminum. In various embodiments, the first metallic material is a coating formed on a portion of the body of the implantable device. In various embodiments, the body of the implantable device defines a cavity within which the first feature is retained. The implantable device may further include a cell growth promoting factor coupled to the body, a cell growth inhibiting factor coupled to the body, and/or a therapeutic agent coupled to the body.

Also disclosed herein, according to various embodiments, is another implementation of an implantable device configured to affect a localized pH at an implantation site. This implementation of the implantable device includes, according to various embodiments, a body, a first feature, and a second feature. The body comprises a base material, the first feature is coupled to the body and comprises a first metallic material, and the second feature is coupled to the body and comprises a second metallic material, according to various embodiments. The first metallic material may have a lower electrode potential than the second metallic material such that the first metallic material is configured to be preferentially oxidized over the second metallic material.

In various embodiments, the body is a structural scaffolding of the implantable device, the first feature is coupled to a first end of the structural scaffolding, and the second feature is coupled to a second end of the structural scaffolding. In various embodiments, the implantable device comprises an electron pathway that extends between the first feature and the second feature. The electron pathway may comprise the structural scaffolding itself (i.e., the base material is electrically conductive) or the electron pathway may comprise an electrically conductive wire extending between the first feature and the second feature (e.g., a wire coiled around the structural scaffolding).

In various embodiments, the implantable device further includes at least one of a cell growth promoting factor and a cell growth inhibiting factor coupled to the body of the implantable device. In various embodiments, the implantable device further includes a therapeutic agent coupled to the body of the implantable device.

Also disclosed herein, according to various embodiments, is an inferior vena cava filter comprising a body, a first feature, and a second feature. The body comprises a base material, the first feature is coupled to the body and comprises a first metallic material, and the second feature is coupled to the body and comprises a second metallic material. According to various embodiments, the first metallic material has a lower electrode potential than the second metallic material such that the first metallic material is configured to be preferentially oxidized over the second metallic material.

In various embodiments, the body of the inferior vena cava filter comprises a hub and a plurality of legs extending from the hub. The first feature may be coupled to at least one leg of the plurality of legs and the second feature may be coupled to the hub. The base material of the body may be electrically conductive, such that an electron pathway comprises the at least one leg of the plurality of legs (i.e., the electron pathway is the body of the filter). In various embodiments, the electron pathway is an electrically conductive wire that extends between the first feature and the second feature. The electrically conductive wire may be coiled around the at least one leg of the plurality of legs.

The forgoing features and elements may be combined in various combinations without exclusivity, unless otherwise expressly indicated herein. These features and elements, as well as the operation of the disclosed embodiments, will become more apparent in light of the following description and accompanying drawings.

### BRIEF DESCRIPTION OF THE DRAWINGS

FIG. 1 is a schematic block diagram of an implantable device having a first feature coupled to a body of the implantable device, in accordance with various embodiments;

FIG. 2 is a schematic block diagram of an implantable device having a first and second feature coupled to a body of the implantable device, in accordance with various embodiments;

FIG. 3 is a schematic block diagram of an implantable device having a first feature and a cell growth factor coupled to the body of the implantable device, in accordance with various embodiments;

FIG. 4 is a schematic block diagram of an implantable device having a first feature, a second feature, and a cell growth factor coupled to the body of the implantable device, in accordance with various embodiments;

FIG. 5 is a schematic block diagram of an implantable device having a first feature, a cell growth factor, and a therapeutic agent coupled to the body of the implantable device, in accordance with various embodiments;

FIG. 6 is a schematic block diagram of an implantable device having a first feature, a second feature, a cell growth factor, and a therapeutic agent coupled to the body of the implantable device, in accordance with various embodiments;

FIG. 7 is a schematic depiction of an inferior vena cava filter implanted within an inferior vena cava of a patient, in accordance with various embodiments;

FIG. 8 is a graph showing pH vs time for incubated implantable devices, in accordance with various embodiments; and

FIG. 9 is a graphic showing various properties of an implantable device that can affect the localized pH at the implantation site, in accordance with various embodiments.

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

### DETAILED DESCRIPTION

The detailed description of exemplary embodiments herein makes reference to the accompanying drawings, which show exemplary embodiments by way of illustration. Although these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and is not limiting.

Disclosed herein, according to various embodiments, are devices, systems, and methods for modulating the localized pH at the implantation site of implantable devices. By controlling, modulating, or otherwise adjusting the localized pH, various benefits can be achieved, such as controlling cell proliferation. The implantable devices disclosed herein may facilitate spatial and/or temporal control of cellular proliferation, such that growth processes, such as endothelialization, may be selectively inhibited at one part of the device and/or during one period of time, and selectively promoted at another part of the device and/or during another period of time.

Though numerous details and examples are included herein pertaining to pH modulation and the resultant cell proliferation (either promoted cell growth or inhibited cell growth) for intravascular and/or intracardiac devices (e.g., IVC filters, vascular stents, vascular stent grafts, cardiac pacing lead wires, etc.), the scope of this disclosure is not necessarily limited to such applications. That is, the embodiments and details of the implantable devices disclosed herein may be utilized for non-vascular, non-cardiac implementations.

Further, the implantable devices disclosed herein may have various other benefits (other than controlling cell growth), such as delivery, activation, and augmented biocompatibility of therapeutic agents. Still further, the implantable devices of the present disclosure may be utilized to control mitogenesis, control cellular adhesion, facilitate wound healing, inhibit infections, treat endometriosis (e.g., intrauterine devices), suppress tumors via inhibition of angiogenesis, and the like. The details of the present disclosure can also be utilized as laboratory tools for scientific research, such as bioreactors, and can be incorporated into cellular manufacturing processes.

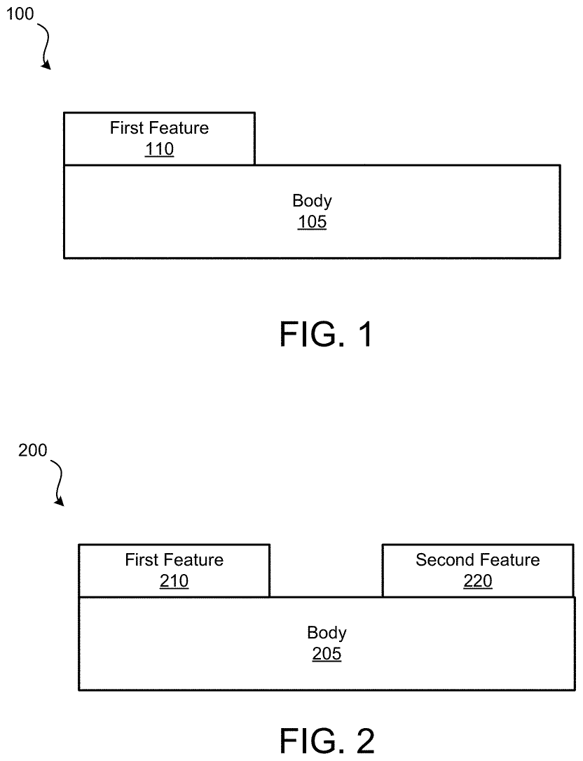

FIGS. 1-6 are schematic block diagrams of implantable devices 100, 200, 300, 400, 500, 600, in accordance with various embodiments of the present disclosure. The implantable devices 100, 200, 300, 400, 500, 600 of FIGS. 1-6 are shown schematically, and thus these figures are not necessarily intended to represent the physical appearance or physical structure of the implantable devices. Generally, the implantable device of the present disclosure include a body (e.g., a structural scaffolding of the implantable device, such as the hub and legs of an IVC filter) having one or more features coupled thereto. The implantable device may have dissimilar metallic features configured to induce galvanic redox reactions to modulate the localized pH. As used herein, the terms “dissimilar metallic features” or “dissimilar metal” refer to any two metallic materials that form a galvanic cell when the implantable device is installed/implanted in a patient such that both metallic materials are in electrolytic communication with each other via a conductive medium (e.g., blood, tissue, etc.). Examples of “dissimilar metals” include, by way of non-limiting example, any two metals selected from the group consisting of gold, platinum, copper, aluminum, barium, lithium, manganese, tin, silver, iron, zinc, magnesium, zirconium, and alloys thereof, among others.

In various embodiments, and with reference to FIG. 1, the implantable device 100 includes a body 105 and a first feature 110. The body 105 comprises a base metallic material and the first feature comprises a first metallic material, according to various embodiments. The first metallic material of the first feature 110 has a lower electrode potential (i.e., a more negative electrode potential) than the base metallic material of the body 105, according to various embodiments. That is, the first metallic material of the first feature 110 may be anodic and thus may be configured to be preferentially oxidized over the base metallic material of the body (which is cathodic).

In various embodiments, and with reference to FIG. 2, the implantable device 200 has a body 205, a first feature 210, and a second feature 220. The body 205 is made from a base material, which may not be metallic, and the first feature 210 and the second feature 220 are coupled to the body and comprising a first metallic material and a second metallic material, respectively. The first metallic material of the first feature 210 has a lower electrode potential than the second metallic material of the second feature 220, and thus the first metallic material of the feature 210 is configured to be preferentially oxidized over the second metallic material of the second feature 220, according to various embodiments. Thus, the body 105 of the implantable device 100 of FIG. 1 is metallic and cathodic and forms a galvanic cell with the anodic first feature 110 while the body 205 of the implantable device 200 of FIG. 2 does not have to be metallic because the second feature 220 is cathodic and the first feature 210 is anodic. As used herein, the term “galvanic cell” generally refers to an electrochemical cell that generates ions from a spontaneous redox reaction taking place at the surface of the metallic materials.

As briefly mentioned above, the body of the implantable device may provide physical structure and shape to the implantable device. That is, the body of the implantable device may provide the structural scaffolding of the implantable device, and/or may provide other features unrelated to the galvanic cell and pertaining to the primary function of the implantable device. For example, the implantable device may have a primary function, such as blood clot filtering in the case of an IVC filter, and the galvanic action of the implantable device may provide a secondary/additional function. In other embodiments, however, primary function of the implantable device may be pH modulation via galvanic action.

The one or more galvanic features may be affixed, attached, or otherwise retained to the body. For example, the first and/or second features of the implantable device may take the form of a coating applied to the body of the implantable device. For example, the first and/or second features may be coatings applied via plasma vapor deposition, sputtering, electroplating, dipping, or the like. In various embodiments, the first and second features may be applied via sewing, weaving, molding, gluing, stapling, brazing, soldering ultrasonic welding, laser welding, and/or additive manufacturing, among others. In various embodiments, the first and/or second features may be enclosed in a cavity defined by the body of the implantable device. For example, the structure of the body may be designed to contain reservoirs of galvanic material that, over time, is consumed. The material of these features/reservoirs can be changed, and the location and/or size of the features/reservoirs may be customized to fit a specific application and/or to produce a desired, localized pH environment in the vicinity of the implantable device. In various embodiments, an existing implantable device may be retro-fit with the first and/or second features, or the methods mentioned above may be utilized to manufacture a new product having these features.

Embodiments according to the present disclosure include methods and devices that are useful in controlling the cellular proliferation, in both space and time, surrounding the implantable devices. More specifically, methods and devices that control (either promote or retard) cellular proliferation surrounding one or more parts or surfaces of the implantable medical device are provided. One embodiment provides methods and devices useful for minimizing post-deployment endothelial cell proliferation on an IVC filter or other intravascular or intracardiac device, and for spatially and/or temporally controlling endothelialization of other implantable medical devices.

Embodiments according to the present disclosure offer these advantages by strategic placement/coating of metal features on the intravascular device. Specifically, the metal features form a galvanic cell comprising all or part of a medical device, or features affixed to a medical device, thus enabling regulation of the pH of the environment surrounding the device or part of the device to inhibit or promote cell proliferation. In one embodiment, an intravascular device comprises a galvanic cell formed from two or more dissimilar metal features, the galvanic cell inhibiting endothelial cell proliferation about the entire surface of the intravascular device. In another embodiment, an intravascular device comprises a galvanic cell formed from one metal that forms a galvanic cell with ions in the blood, thereby inhibiting endothelial cell proliferation about a surface of the intravascular device.

In embodiments of the present disclosure, an implantable medical device comprises a base, a first metal feature, and optionally a second metal feature, the first and second metal features form a galvanic cell surrounding at least a part of the implantable medical device. The first and optional second metal features may, but need not, comprise metals selected from the group consisting of gold, platinum, copper, aluminum, barium, lithium, manganese, tin, silver, iron, zinc, magnesium, zirconium, and combinations thereof. The part of the implantable medical device surrounded by the galvanic cell may be, by way of non-limiting example, a distal end, a proximal end, distal and proximal ends, or the entire implantable device.

The present disclosure also provides methods for controlling endothelialization of an intravascular or intracardiac device, comprising providing one, two, or more metal features that form a galvanic cell; and implanting the intravascular or intracardiac device in a blood vessel or heart of an animal, whereby the galvanic cell maintains a pH environment in situ about at least a part of the intravascular or intracardiac device that inhibits or promotes endothelial cell proliferation. It should be noted that, due to the ionic content of the blood, a single metal feature could also be used to alter the pH of the environment.

In various embodiments, the implantable device, which may be referred to as an in-situ galvanic placed within a body of a patient, creates a local, pH-controlled environment. Based on selection of proper galvanic materials, it is possible to introduce higher pH around the regions of the implantable device, thus inhibiting uncontrolled cell growth. Additionally, controlling pH allows for controlling of endotheliaziation as opposed to inhibition. For example, moderately alkaline environments can allow initial proliferation, enough to provide stability, but inhibit spreading and overgrowth associated with IVC filter retrieval complications.

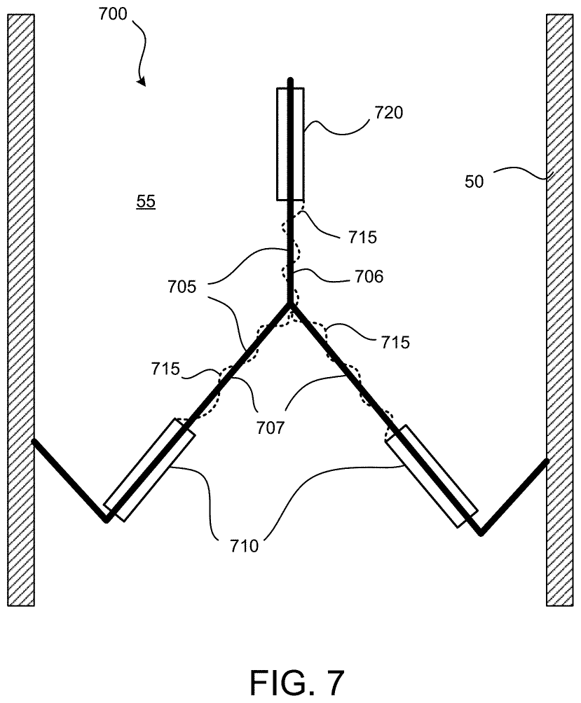

In various embodiments, and with reference to FIG. 7, an IVC filter 700 is provided. The IVC filter 700 may include a body 705 made from a base material. The IVC filter 700 is depicted in FIG. 7 in an installed position with the inferior vena cave 50. The IVC filter 700 may also include one or more first features 710 coupled to the body 705 and one or more second features 720 coupled to the body 705. As explained before, the first metallic material of the first feature 710 may have a lower electrode potential than the second metallic material of the second feature 720, such that the first metallic material of the first feature 710 is configured to be preferentially oxidized. Said differently, the first feature 710 forms the anode of the galvanic cell and the second feature 720 forms the cathode of the galvanic cell. For example, the first feature 710 may comprise magnesium and the second feature 720 may comprise copper hydroxide.

In various embodiments, the body 705 of the IVC filter 700 includes a hub 706 and a plurality of legs 707 extending from the hub 706. The first feature(s) 710 may be coupled to at least one leg of the plurality of legs 707, though it may be advantageous for each leg to have the first feature coupled thereto, and the second feature 720 may be coupled to the hub 706. The body 705 may be made from a metallic material that is sufficiently electrically conductive, and thus an electron pathway is formed by the body 705 and extends between the first features(s) 710 and the second feature 720 (e.g., the electron pathway may be formed by the at least one leg 707 and the hub 706 of the body 705). In other embodiments, the body 705 may not be made from an electrically conductive material, and thus the implantable device may include an electrically conductive wire 715 to provide the necessary electron pathway between the first and second features 710, 720. The electrically conductive wire 715 may be coiled around the at least one leg of the plurality of legs to extend between the two features 710, 720.

In various embodiments, depletion of electrons from the anodic first feature 710 will cause dissemination of hydroxyl anions on the cathodic end (second feature 720) of the implantable device. Accumulation of hydroxyl groups will create a local increase in pH, according to various embodiments. As a result, the basic/alkaline environment suppresses cellular proliferation. The kinetics of the galvanic cell will depend on the type of galvanic metals used, the electrical resistance (conductivity) of the electron pathway, as well as the electrolytic material (i.e., blood 55). Returning to the example mentioned above where the first feature 710 is made from magnesium and the second feature 720 is made from copper hydroxide, the standard voltage reduction potential of the magnesium is −2.37 Volts, thus creating a driving force for the reduction of the copper alloy and corresponding generation of hydroxyl groups. Exemplary half-reactions and the corresponding overall reaction are provided below:

Anode: Mg(s)↔Mg²⁺+2e⁻

Cathode: Cu(OH)₂+2e⁻↔Cu(s)+2OH

Complete Reaction: Mg(s)+Cu(OH)₂↔Mg²⁺+Cu(s)+2OH⁻¹

In other embodiments of the present disclosure, the body of the implantable device is partially coated with gold or another coating metal. In various embodiments, both a distal end and a proximal end of the device are coated. In another embodiment, only the distal end, or only the proximal end, of the device is coated. The length of either the distal end or the proximal end that is coated with the coating metal may, by way of non-limiting example, be between about 1 μm and about 50 mm.

Embodiments according to the present disclosure offer these advantages by strategic placement of at least one metal, or two or more dissimilar metal features, of the implantable medical device. Specifically, the one metal, or two or more dissimilar metal features, form a galvanic cell comprising all or part of the implantable medical device, thus enabling regulation of the pH of the environment surrounding the device or part of the device to inhibit or promote endothelial cell proliferation. In one embodiment, an IVC filter comprises a galvanic cell formed from two or more dissimilar metal features, the galvanic cell inhibiting endothelial cell proliferation about the entire surface of the IVC filter. In another embodiment, an IVC filter comprises a galvanic cell formed from one metal that, in contact with ions present in the blood of a patient, forms the galvanic cell inhibiting endothelial cell proliferation about the entire surface of the IVC filter.

Galvanic cells on implantable medical devices of the present disclosure provide a microenvironment inside the body of an animal in which pH is spatially and/or temporally controlled to selectively inhibit or promote endothelial cell proliferation at a desired portion of the medical device for a desired time. The galvanic cell may be designed to control pH at a specific level by selecting an appropriate geometry, quantity, and metal for each of the one, two, or more metal features. The anode and the cathode of the galvanic cell may be in direct physical contact, or the anode and the cathode may be separated by and exposed to a common electrically conductive medium including, but not limited to, fluids and tissues of the animal itself

Implantable medical devices of the present disclosure may comprise a base material, and metal that forms either an anode or a cathode when in contact with ions in the blood of a patient. The metal may be, by way of non-limiting example, selected from the group consisting of stainless steels, cobalt-chromium alloys, titanium alloys, nickel-titanium alloys, tantalum, titanium, Elgiloy®, gold, platinum, copper, aluminum, barium, lithium, manganese, tin, silver, iron, zinc, magnesium, and zirconium.

In another embodiment of the present disclosure, the base material used to form the structural scaffolding of the implantable medical device is cathodic and is coated with an anodic coating metal. By way of non-limiting example, the structural scaffolding of the implantable medical device may be made using a shape-memory metal such as nitinol (an alloy of nickel and titanium) as the base material. The structural scaffolding is manufactured according to methods known in the art. The structural scaffolding is then coated with a coating metal, such as magnesium, zinc, or iron, using methods known to those skilled in the art such as plasma vapor deposition, sputtering, electroplating, dipping, and the like. The coating metal may coat the entire device surface or may be limited to one or more ends, as described above.

In other embodiments, an implantable medical device made in accordance with the teachings of the present disclosure comprises galvanic cells affixed to a luminal wall-contacting surface of the device. The galvanic cells of the present disclosure can be affixed to the device using any means known to those in the art, including, by way of non-limiting examples, sewing, weaving, molding, gluing, stapling, brazing, soldering, and welding. In one embodiment of the present disclosure, a galvanic cell comprising stainless steel as the base metal and zinc as the active metal is affixed to the luminal wall-contacting surface of an IVC filter using a biocompatible cyanoacrylate adhesive. In another embodiment of the present disclosure, the galvanic cell comprises a base metal selected from the group consisting of stainless steel, cobalt-chromium alloys, nickel-titanium alloys, tantalum, titanium, Elgiloy®, and combinations thereof and the coating metal is selected from the group consisting of gold, platinum, silver, iron, zinc, magnesium, zirconium and combinations thereof.

Another implantable medical device of the present disclosure may comprise a base material, an anode made of a first metal, and a cathode made of a second metal. Each of the first and second metals may, by way of non-limiting example, be selected from the group consisting of stainless steels, cobalt-chromium alloys, titanium alloys, nickel-titanium alloys, tantalum, titanium, Elgiloy®, gold, platinum, copper, aluminum, barium, lithium, manganese, tin, silver, iron, zinc, magnesium, zirconium, and combinations thereof; those of ordinary skill in the art will understand how to select first and second metals that are “dissimilar metals,” as that term is used herein, and that are otherwise suitable for a desired application.

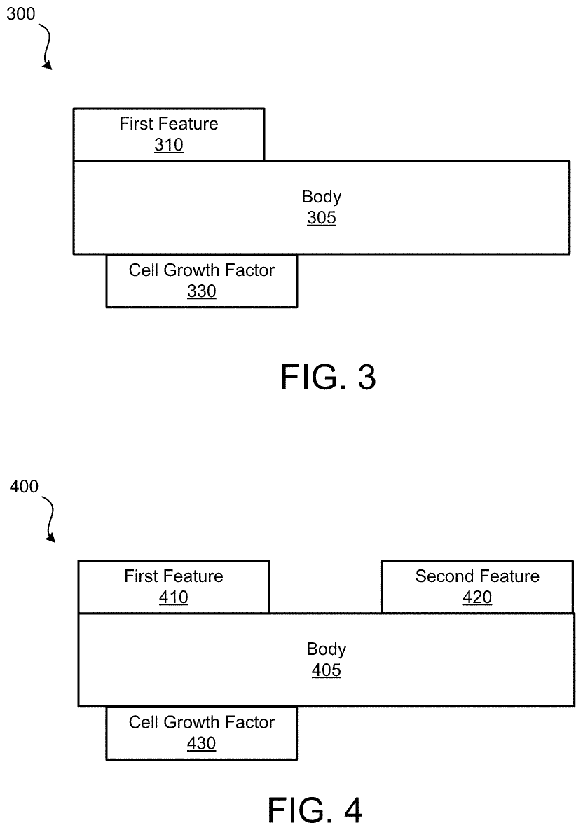

In various embodiments, and with reference to FIGS. 3 and 4, the implantable device may include one or more cell growth factors coupled to the body. That is, the implantable device 300, which may have a body 305 and a first feature 310 similar to the corresponding features described above with reference to FIG. 1, may also include a cell growth factor 330 coupled to the body 305. Similarly, the implantable device 400, which may have a body 405, first feature 410, and second feature 420 similar to the corresponding features described above with reference to FIG. 2, may also include a cell growth factor 430 coupled to the body 405. The cell growth factor(s) may be affixed, attached, or deposited according to the methods and procedures described above with reference to the first and second features. The cell growth factor may be a promoting factor or an inhibiting factor. For example, a cell growth promoting factor (CGPF) may be disposed on one or more of the structural scaffolding portions of the body.

The cell growth factor promotes growth of cells from the vascular endothelium around the structural scaffolding portions. Other embodiments according to the present disclosure provide mechanisms to further stimulate endothelialization around a portion of an intravascular device by providing a cell growth promoting factor on all or a subset of structural scaffolding portions, other than those located at the ends. Exemplary CGPFs suitable for use in the present disclosure include, but are not limited to, vascular endothelial growth factor (VEGF), platelet-derived growth factor (PDGF), platelet-derived epidermal growth factor (PDEGF), fibroblast growth factors (FGFs) including acidic FGF (also known as FGF-1) and basic FGF (also known as FGF-2), transforming growth factor-beta (TGF-β), and platelet-derived angiogenesis growth factor (PDAF). The combination of the CGPFs and the galvanic coating provides spatial and/or temporary control of endothelialization of the surfaces of the intravascular device. It is to be understood that cell growth inhibiting factors may be provided instead of, or in addition to, CGPFs on various portions of the intravascular device, providing a further degree of control of endothelialization.

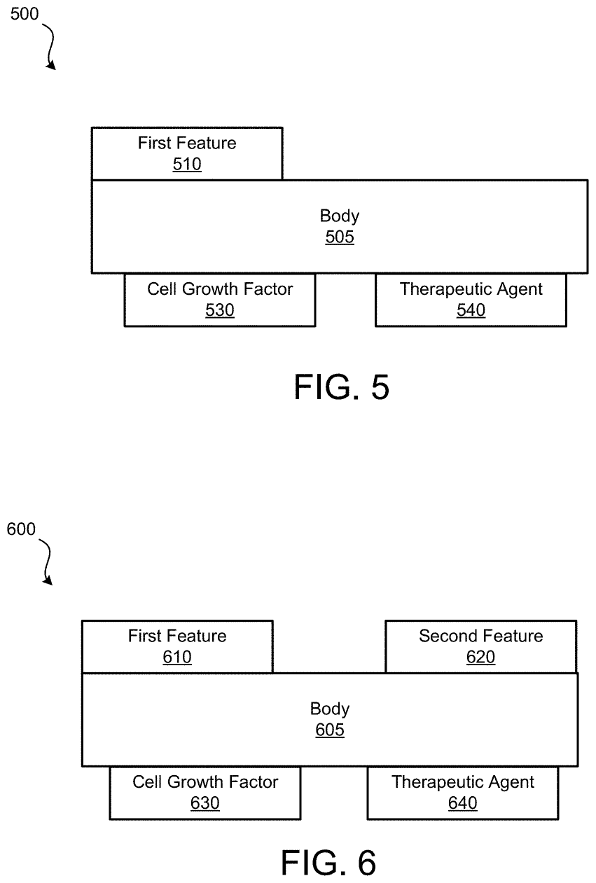

In various embodiments, and with reference to FIGS. 5 and 6, the implantable device may include one or more therapeutic agents coupled to the body. That is, the implantable device 500, which may have a body 505 and a first feature 510 similar to the corresponding features described above with reference to FIGS. 1 and 3, may also include a therapeutic agent 540 coupled to the body 505. Similarly, the implantable device 600, which may have a body 605, first feature 610, and second feature 620 similar to the corresponding features described above with reference to FIGS. 2 and 4, may also include a therapeutic agent 640 coupled to the body 605. The therapeutic agents may be implemented in conjunction with cell growth factors or in place of cell growth factors. That is, the implantable device may be implemented without cell growth factors and just one or more therapeutic agents. The therapeutic agent(s) may be affixed, attached, or deposited according to the methods and procedures described above with reference to the first and second features. The implantable devices may facilitate in-situ delivery of the therapeutic agents. In various embodiments, the pH modulation caused by the galvanic cell of the implantable device facilitates the delivery, activation, bioavailability, and/or biocompatibility of therapeutic agents.

Examples of suitable therapeutic agents include, but are not limited to, anti-thrombotic agents (e.g., prostacyclin, heparin, and salicylates), thrombolytic agents (e.g., streptokinase, urokinase, tissue plasminogen activator (TPA), and anisoylated plasminogen-streptokinase activator complex (APSAC)), vasodilating agents (e.g., nitrates and calcium channel blocking drugs), anti-proliferative agents (e.g., colchicine), alkylating agents, intercalating agents, antisense oligonucleotides, ribozymes, aptomers, growth modulating factors (e.g., interleukins), transformation growth factor β and congeners of platelet derived growth factor, monoclonal antibodies directed against growth factors, anti-inflammatory agents, modified extracellular matrix components and receptors therefor, and lipid and cholesterol sequestrants.

As mentioned above, though the foregoing disclosure has been directed primarily to intravascular and intracardiac devices, those of ordinary skill in the art will understand that the disclosure is applicable to any implantable medical device for which undergrowth or overgrowth of cells on the device is a concern. By way of non-limiting example, non-vascular stents, orthopedic hardware, and depot formulations for extended release of pharmaceuticals may benefit from modification according to the methods and devices disclosed herein, and such applications are within the scope of the present disclosure.

### EXAMPLE 1

### In Vitro Testing of IVC Filter Components

Prototype devices were constructed using Magnesium and Copper bound to stainless steel wire with cyanoacrylate. These devices were placed in either 1× Phosphate Buffer Saline or Dulbecco's Modified Eagle Media. pH changes were observed at time 0 and time +4 hours.

The experiment was repeated using phosphate buffered saline with 15 drops of a universal indicator solution. Devices were placed in glass scintillation vials and kept there for several days. After 48 hours, pH was measured at 7.5.

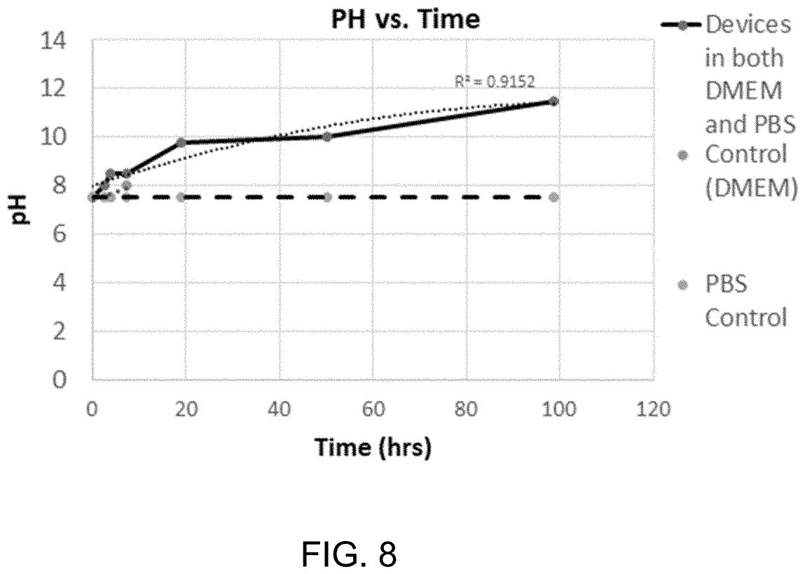

Results from all experiments are summarized in FIG. 8, which shows pH as a function of time. Additional observations were made, including the formation of bubbles around devices indicating generation of hydroxyls. Also, faint regions of color were observed around the tips of devices indicating regional gradients in pH. Bubbles were observed, indicating generation of hydroxyls.

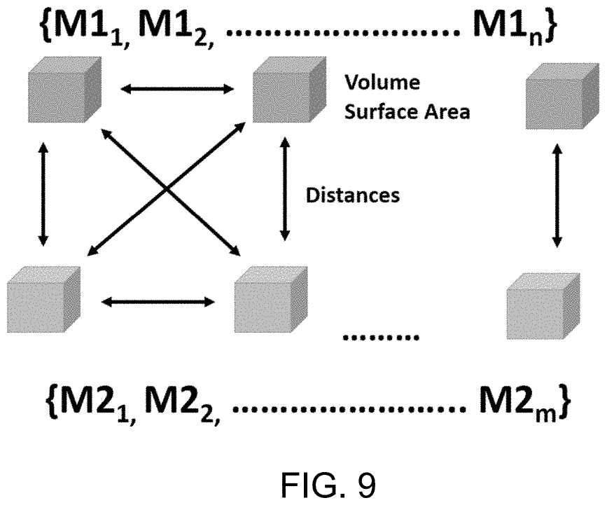

In various embodiments, and with reference to FIG. 9, various properties of an implantable device can affect the pH at the implantation site. For example, the spacing between the galvanic features, the pattern of the galvanic features, the number of galvanic features, the material volume of the galvanic features, and the surface area of the galvanic features, among other properties, can be customized and adjusted in order to produce a desired pH environment at the implantation site. These properties of the implantable device may also be configured to produce a specific pH distribution/gradient at the implantation site, thereby providing a spatial and/or temporal distribution of ions that are specifically tailored for the implantation site.

In various embodiments, the present disclosure also provides a method of controlling a localized pH at an implantation site of a device. The method may include designing and manufacturing the implantable device to have specific properties in order to generate a specific pH distribution, as described above. The method may include strategically placing dissimilar metals to influence cellular proliferation. In various embodiments, the method may also include utilizing one or more scanning/sensing technologies to determine the extent of galvanic corrosion of the dissimilar metals to determine if removal/replacement is warranted.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An implantable device configured to affect a localized pH at an implantation site, the implantable device comprising:
a body of the implantable device, the body comprising a base metallic material; and
a first feature coupled to the body of the implantable device, the first feature comprising a first metallic material;
wherein the first metallic material has a lower electrode potential than the base metallic material such that the first metallic material is configured to be preferentially oxidized over the base metallic material.

2. The implantable device of claim 1, wherein the first feature is anodic and the body is cathodic.

3. The implantable device of claim 1, wherein the first metallic material comprises at least one of magnesium, zinc, and aluminum.

4. The implantable device of claim 1, wherein the first metallic material is a coating formed on a portion of the body of the implantable device.

5. The implantable device of claim 1, wherein the body of the implantable device defines a cavity within which the first feature is retained.

6. The implantable device of claim 1, further comprising a cell growth promoting factor coupled to the body of the implantable device.

7. The implantable device of claim 1, further comprising a cell growth inhibiting factor coupled to the body of the implantable device.

8. The implantable device of claim 1, further comprising a therapeutic agent coupled to the body of the implantable device.

9. An implantable device configured to affect a localized pH at an implantation site, the implantable device comprising:
a body of the implantable device, the body comprising a base material;
a first feature coupled to the body of the implantable device, the first feature comprising a first metallic material; and
a second feature coupled to the body of the implantable device, the second feature comprising a second metallic material;
wherein the first metallic material has a lower electrode potential than the second metallic material such that the first metallic material is configured to be preferentially oxidized over the second metallic material.

10. The implantable device of claim 9, wherein:
the body is a structural scaffolding of the implantable device;
the first feature is coupled to a first end of the structural scaffolding; and
the second feature is coupled to a second end of the structural scaffolding.

11. The implantable device of claim 10, wherein:
an electron pathway extends between the first feature and the second feature; and
the electron pathway comprises the structural scaffolding such that the base material is electrically conductive.

12. The implantable device of claim 10, wherein:
an electron pathway extends between the first feature and the second feature; and
the electron pathway comprises an electrically conductive wire extending between the first feature and the second feature.

13. The implantable device of claim 12, wherein the electrically conductive wire is coiled around the structural scaffolding.

14. The implantable device of claim 9, further comprising at least one of a cell growth promoting factor and a cell growth inhibiting factor coupled to the body of the implantable device.

15. The implantable device of claim 9, further comprising a therapeutic agent coupled to the body of the implantable device.

16. An inferior vena cava filter comprising:
a body comprising a base material;
a first feature coupled to the body, the first feature comprising a first metallic material; and
a second feature coupled to the body, the second feature comprising a second metallic material;
wherein the first metallic material has a lower electrode potential than the second metallic material such that the first metallic material is configured to be preferentially oxidized over the second metallic material.

17. The inferior vena cava filter of claim 16, wherein the body comprises a hub and a plurality of legs extending from the hub, wherein the first feature is coupled to at least one leg of the plurality of legs and the second feature is coupled to the hub.

18. The inferior vena cava filter of claim 17, wherein the base material of the body is electrically conductive, wherein an electron pathway extends between the first feature and the second feature, wherein the electron pathway comprises the at least one leg of the plurality of legs.

19. The inferior vena cava filter of claim 17, wherein an electron pathway extends between the first feature and the second feature, wherein the electron pathway comprises an electrically conductive wire extending between the first feature and the second feature.

20. The inferior vena cava filter of claim 19, wherein the electrically conductive wire is coiled around the at least one leg of the plurality of legs.
